(19) United States
(12) Patent Application Publication
(10) Pub. No.: US 2011/0224502 A1
(43) Pub. Date: Sep. 15, 2011

(54) METHOD AND APPARATUS FOR DIAGNOSIS AND TREATMENT

(76) Inventor: Ewa Herbst, Edgewater, NJ (US)

(21) Appl. No.: 12/485,855

(22) Filed: Jun. 16, 2009

Related U.S. Application Data
(60) Provisional application No. 61/129,285, filed on Jun. 16, 2008.

(51) Int. Cl. A61B 5/00

(52) U.S. Cl. 600/301

(57) ABSTRACT

An apparatus and method for performing multiple measurements and diagnoses simultaneously, in terms of body function, have a data processing element, a data acquisition module connected to the processing element, an optional treatment output module connected to the processing element, a user control module connected to the processing element, optionally a user display system connected to the processing element, and wherein the data acquisition module sends a plurality of measurement data simultaneously, in terms of body functions, to the data processing element. The processing element provides a plurality of outputs which can be made available to the user or combined in the diagnostic module. This in turn using a treatment algorithm in the treatment module can affect the patient through a feedback mechanism. In response thereto, the data processing element generates treatment information needed by the treatment output module. In one embodiment, the data acquisition module sends multiple different measurement data relating to a single disease to the data processing element. The method and apparatus can also monitor and diagnose treatment of a disease by simultaneously, in terms of body functions, measure, using a single data acquisition module, multiple parameters relating to the diagnosis or treatment of the disease; and in response to said measured parameters, determine the diagnosis or treatment of the disease; effecting a treatment regimen based on the determined diagnosis or treatment; and in response to further measurements of the parameters, determining an effectiveness of the treatment, and modifying, as necessary, the treatment regimen.

### CROSS-REFERENCE TO RELATED APPLICATIONS

This application is claims priority to and the benefit of U.S. Provisional Application No. 61/129,285, filed Jun. 16, 2008. The entire contents of each of the above-identified applications are hereby incorporated by reference herein.

### FIELD OF THE INVENTION

The invention relates generally to analysis methods and equipment, and in particular to measurement, diagnostic, and treatment equipment able to perform any or all of the functions substantially simultaneously or in a prescribed order. It allows not only for immediate testing but also for long term monitoring of the disease, and for treatment in response to such monitoring, as well as for monitoring of treatment efficacy, which can have importance both for personalized medicine and for drug discovery.

The equipment will allow a lab or doctor not only to diagnose the patient (human or non-human (for example, a cow, sheep, horse, etc.)) but also to approach each patients needs individually (effecting truly personalized medicine) in order to:
1) be able to identify a sub-population of patients who may not be appropriate candidates for a specific treatment, either due to a predicted lack of treatment efficacy, or due to potentially significant side effects for their biological make-up;
2) allow for appropriate individual treatment dose and delivery scheme, based not only, for example, on the patient's age and weight, but also on biomarkers and other measured analytes; and
3) allow for a combination of drugs (in appropriate doses), based on the individual patient's test results.

### BACKGROUND OF THE INVENTION

The medical device field for measurement, analysis, and treatment of the human (and non-human) condition has grown substantially over the past years as the ability to build customized equipment, easily and quickly using specialized chips has enabled both large and small companies to enter the field. Of particular interest has been the use of so-called “biomarkers”, each of which can be defined to represent a specific measurement or series of measurements, representative of a specific condition or function of the human body. Such biomarkers typically relate to a biological condition, state, or function, using not measurements of seemingly unrelated parameters such as blood gases (e.g., pO₂, pCO₂), pH, electrolytes, temperature, measured bodily electrical signals (e.g., EKG, EEG, EMG), etc.

Substantial quantities of data relating to biomarkers and other parameters regarding the human condition such as blood gases, pH, electrolytes, temperature, electrical signals and the like have been collected for many specific diseases of the body. Also automatic test equipment has been marketed and has been, typically, measurement driven. Equipment is available for measuring pH, oxygen, and temperature at various parts of the body, and various biological measurement schema which are intended to measure, for example, sugar levels, blood counts, the presence of various genes, proteins, acids, etc., and so on are also available. Such equipment is available from many different vendors and provides in many cases, excellent results for the measurement for which they were designed. It is then, typically, up to the doctor or an automated analysis device, which is used by the lab or the doctor and into which selected data is provided, as requested by the doctor, to provide a diagnosis of the patient.

Similar advances are being made in connection with non-human measurement and analysis, as well as in the measurement and analysis of environmental “parameters” (for example, quality of water) in an effort to improve and automate the analysis and resulting diagnosis and conclusions relating to the input data.

### SUMMARY OF THE INVENTION

Embodiments of the invention relate to the design, use, and manufacture of a diagnostic apparatus able to perform multiple diagnostics, on the same or different diseases, within a short period of time, in terms of human body functions, instantaneously. The diagnostic equipment can use a front end, commercially available, to generate signals representative of various bio-markers, as well as signals, from the same or different sensing device (proprietary or commercial) representative of other indicia, for example of the body, not considered biomarkers as that term is currently used. Multiple discrete sensing modules or a single sensing module can be used as the front end to receive sensor signals, and the signals can be presented simultaneously or serially, to the sensing module(s), and then to a processing unit (for example, a digital signal processor (DSP)) which can be integrated into or separate from the sensing modules/devices or sensors. The analysis unit, that is, the processing unit, can have varying degrees of complexity, from a totally flexible research analysis function, incorporating user controlled, powerful processing, to one or more customized units which may or may not be user controllable, with their output being either a user defined visual, printed, audio, digital, or other presentation or signal for controlling the delivery of diagnostic information or signals, for example, for controlling the delivery of treatment directly or indirectly to or for the patient. The entire system can also be manufactured on a chip which may be a custom chip, for example, an Application Specific Integrated Circuit (ASIC), a chip preset for a class of applications, for example, an Application Specific Standard Product (ASSP), etc. The chip can be embedded in a patient, as part of a totally self-sufficient implantable device. It can also be connected either wired or wirelessly to an external viewing and communication device (desktop, handheld, or PC) allowing for a manual control of all measured parameters and treatment regimens, a data dump to the external memory or PC, and additional analysis. It can be also wired or wirelessly connected to an analysis equipment for diagnostic processing, and can be integrated into a complete stand-alone system connected to receive data from internal or external sources/sensors. The output of the chip can be interfaced with other electronic equipment, through any appropriate protocol, including secure protocols for addressing privacy concerns. While the apparatus of various embodiments can be manufactured for specific applications, that is, to check selected biomarkers and other signals for a specific disease or diseases, or other conditions, the apparatus can also interrogate a multiplicity of biomarkers, across several diseases to be examined, viewed, treated, etc., in order to provide and improve treatment outcomes due to the immediate feedback nature of the apparatus. Further, a standard set of biomarkers fitting a profile for discovering or treating a specific disease can, if appropriate, have added to it non-biomarker indicia which have the effect of improving the accuracy of the diagnosis.

Tests comprising of these markers for, for example, coronary heart disease may include cholesterol (HDL and LDL) and triglycerides combined with homocysteine and C-reactive protein (CRP), which is a general marker for inflammation and infection and PLAC testing, which in combination with LDL information is highly predictive of coronary heart disease. In an acute situation, ischemia-modified albumin and blood gases and ions will be of value. Roche Diagnostics, as an example, has a cardiac reader analyzer which allows the quantitative determination of troponin T, myoglobin, D-Dimer and now N-terminal proBNP (NT-proBNP) from a single whole blood sample within minutes.

Breast cancer tests may include CA 15-3 and CA 27.29 to follow-up breast cancer patients for reoccurrence of cancer, while ovarian cancer tests may comprise a variety of markers depending on the type of the tumor. For example, in the case of epithelial tumors, tests may comprise a combination of CA-125 (cancer antigen 125), BRCA-1 and BRCA-2, carcinoembrionic antigen (CEA), galactosyltranferase, and tissue polypeptide antigen (TPA). In the case of germ cell tumors, AFP (alpha feto protein) and quantitative hCG (human chorionic gonadotropin) can be measured, while for stromal tumors, Inhibin would be of interest.

In colon cancer, the fecal immunochemical test (FIT) or an immunochemical fecal occult blood test (iFOBT) are used. Stool DNA test looks for abnormal sections of DNA instead of blood in the stool. If results of those tests are positive, colonoscopy is required. Alternatively novel methods being developed by others rely on probe measurements which would make colonoscopy unnecessary and which could incorporate the invention.

The apparatus according to an embodiment of the invention can also diagnose, and/or treat as well as make measurements of, those bodily indicia which on their face, are uncorrelated to each other. While not obviously thereby interconnected, these measurements may, when performed substantially simultaneously (in body time) to biomarker measurements, provide insight into the nature and the occurrence of diseases as well as provide an advantage in treating diseases by correlating two or more indicia, previously considered to be unrelated.

The additional measurements may include pO₂, pH, temperature, pressure, electrical signals (as noted above), etc. The apparatus, according to one embodiment, can also be connected to the Ethernet or Internet to download the latest protocols, to update different levels of concern in regard to the medical measurement and diagnosis, and to provide additional or different measurements in connection with a study, such as patient data or images, for example, an MRI, CT scan, X ray.

The apparatus provides answers to the analytical questions based on inputs from biomarker and other non-biomarker signals. The analysis is primarily based on the biological/biochemical markers but can be aided by combination with non-biology input measurements such as electrical signals (e.g., EKG, EEG, EMG), chemical, physical, electrochemical, etc. When connected in a feedback configuration, the apparatus can monitor the results of treatment, in real time, to continuously or intermittently adjust and control the treatment delivery.

The invention thus relates to an apparatus for performing multiple diagnostics simultaneously, in terms of body function, having a data processing element, a data acquisition module connected to the processing element, a treatment output module connected to the processing element, a user control module connected to the processing element, a user display system connected to the processing element, and wherein the data acquisition module sends a plurality of measurement data simultaneously, in terms of body functions, to the data processing element, and in response thereto, the data processing element generates treatment information needed by the treatment output module.

In another aspect, the invention relates to a method for monitoring and diagnosing treatment of a disease featuring simultaneously, in terms of body functions, measuring, using a single data acquisition module, at least five parameters relating to the diagnosis or treatment of the disease, where in response to the measured parameters, the method determines the diagnosis or treatment of the disease, and effects a treatment regimen based on the determined diagnosis or treatment.

In response to further measurements of the parameters, the method determines an effectiveness of the treatment, and modifies, as necessary, the treatment regimen.

### DESCRIPTION OF THE DRAWING

Other objects, features and advantages of the invention will be apparent from the following description, in connection with the drawing in which;

FIG. 1 is a functional blocked diagram illustrating one particular embodiment of the invention.

### DETAILED DESCRIPTION

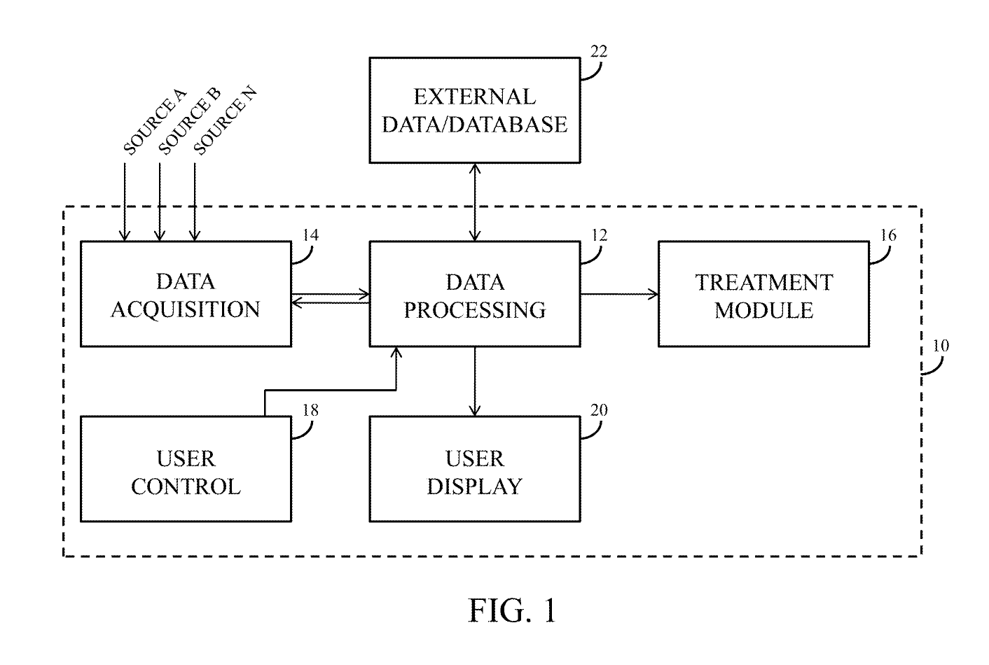

Referring to FIG. 1, a method and apparatus in accordance with aspects of the invention include a system 10 having various modules or partitions. The system 10 can be modeled after/or be the same as, in many ways, the system on a chip described in U.S. patent application Ser. No. 12/113,200, filed on Apr. 30, 2008, titled Method and Apparatus for Configurable Systems on a Chip, the contents of which are incorporated herein by reference in their entirety.

Referring to FIG. 1, again, the data processing element 12, for example a digital signal processor, a custom data processor, or any other type of data processing unit, acts as the central processing module for the system 10. In this embodiment, the processing element 12 connects to a data acquisition module 14, a treatment output module 16, a user control module 18, and a user display system 20. At a high level, in operation, the data processing module 12 receives measurements and data in the form of, for example, biomarker and non-biomarker data from the data acquisition unit, which is operating under the control of the data processing element 12. The data processing element processes that information, using, if appropriate, further information and data received from an external database or other external unit 22, and in response to user commands from a user control module 18 can generate, as required, both user display information for the user display 20 and treatment information as needed for treatment module 16. If the apparatus is embedded in the patient, it can be connected by hard wired cables, or wirelessly, continuously or intermittently, as necessary. In this exemplary configuration, a visual user display, for example, need not be used or provided. It can be, however, connected preferably wirelessly to an external monitoring unit or PC for display of the patients status and for communication and manual control of the system, if required.

The data acquisition module 14 receives data from many different sources, for generating biomarker information as well as non-biomarker data. Biomarkers may be associated for a particular disease, or for a range of diseases, or alternatively for a series of predefined biomarkers as dictated by the user. Methods such as genomics, proteomics, and/or molecular imaging, among other methods, can be used in the generation of the biomarker information. Among specific methods used, variety of spectroscopic methods can be applied, such as fluorescent spectroscopy, mass spectroscopy, which can be used, e.g., for gene expression profiling, Raman spectroscopy and lately Fourier transform infrared spectroscopy (RTIR). The various sources then, illustrated as source A, source B . . . source N, can be sources of data associated directly with measurements made in a patient, such as, for example, temperature, pH, pO₂, etc. as described previously, for the non-biomarker data or data provided directly from the patient or from test results relating to biological functions and useful for determining biomarker information. Such data can be obtained as described in the art, and, more particularly, as described in, for example, applicant's U.S. Pat. Nos. 7,160,241, 6,684,106, 6,021,347, 6,708,066, U.S. patent application U.S. Ser. No. 11/151,967 filed Jun. 14, 2005, and patent application U.S. Ser. No. 12/098,257, filed Apr. 4, 2008, the disclosures of which are hereby incorporated by reference in their entirety.

Once the system operator has indicated which data to acquire and use, or which disease or diseases, or other conditions, to acquire data for, the data processing system then operates upon that data, in connection with its own internal memory as well as memory available to it in the form of external data or a database 22, to generate either user display information in oral, written, or display form, or any other form, needed or required by the user, and/or further, upon user control of user control module 18, the system can provide feedback, for example, to the patient in the form of treatment. Such feedback, in the form of treatment, allows for substantially automatic (or user controlled) feedback to regulate the delivery of drugs, or electronic signals, or other treatment protocols, which may then result in revised data from data acquisition module 14, thereby setting up a feedback control loop operating in response to user control, and, if desired, viewable on user display 20, as processed by data processing element 12 and implemented by the treatment module 16. Such treatment can be automatic (using feedback) and may be that described in the above-identified delivery application U.S. Ser. No. 12/098,257, or in applicant's other related patent applications, such as U.S. Ser. No. 12/113,200 filed on Apr. 30, 2008, the contents of which are incorporated herein, in their entirety, by reference. The system can be set up (including inputs, outputs and feedbacks), data acquisition can be controlled, measurements can be processed, diagnostic or treatment algorithms can be applied and and various communications schemes can be provided by, e.g., the systems described in patent application U.S. Ser. No. 61/119,244, filed on Dec. 2, 2008, the content of which is incorporated herein, in its entirety, by reference.

Thus, treatments can be either manual and/or automatic, and made more patient-related as the system reacts to the responsiveness and effectiveness of the treatments, to modify the treatments. In this regard, the signals from the external sources can be substantially simultaneous with regard to the human body's “time constant” for change and thus, all measurements can be treated as made at the same time, so long as the measurement device does not adversely interact with the measurement itself. In a specific embodiment, the measurements are made in real time and simultaneously. With such a substantial amount of data available to the data processing element 12, the system can effect diagnosis of patient concerns faster, and more accurately than prior systems which were directed to a particular type of measurement and analysis and small group of predetermined biomarkers which may or may not relate to many or any specific diagnoses/diseases. In the current apparatus, as described, the system can either collect a broad range of biomarker and non-biomarker information, substantially and preferably simultaneously as defined herein, in order to process the data and yield a result which relates to a probable diagnosis relevant to a patient's concerns and complaints. Alternatively, if the diagnosis is to relate to a specific disease, for example, breast cancer, ovarian cancer, coronary artery disease, or colorectal cancer, to name a few, a selected group of biomarker and non-biomarker measurements can be made, substantially simultaneously, in order to better diagnose the status of, and define the treatment for, such a disease in the patient.

In operation, then, the system 10 initially operates to monitor and define, based on the inputs available to it, a disease, or provide a diagnosis through a specifically developed diagnostic algorithm. The user defines different inputs, typically available in parallel, usually not from the same sensing device, in a user-friendly manner and environment. The output is either a separate measurement or a set of separate measurements provided for each sensor/biomarker or a combined diagnosis, based on the inputs available, which can be taken to the “next level” wherein a treatment regimen is determined and prescribed. A display is provided illustrating the diagnosis and the basis therefor. Other information is provided to the user. In addition, information can be provided to an internal and/or external database to provide better patient management for future efforts.

The resulting device can be used in different configurations for both standard and acute measurements and treatment, for example, in one configuration for a yearly standard health screening and in another for the emergency room, intensive care, or ambulance use. An additional application area, with possibly specific configuration, could be for emergency situations on passenger air planes and at the airports, where medical personnel may not be available.

As noted above, the invention will also be useful with non-human patients, as well as in measuring and “treating” the environment. In that respect, the specifics of the measurements and their timing may be altered depending upon the specific conditions being tested and treated.

Other objects, features and advantages of the invention will be apparent to those practiced in the field and are within the scope of the invention.

1. An apparatus for performing multiple diagnostics simultaneously, in terms of body function, comprising:
a data processing element;
a data acquisition module connected to the processing element;
a treatment output module connected to the processing element;
a user control module connected to the processing element;
a user display system connected to the processing element; and
said data acquisition module sending a plurality of measurement data simultaneously, in terms of body functions, to said data processing element, and in response thereto, the data processing element generates patient information needed by the treatment output module.

2. The apparatus of claim 1 further comprising:
said data processing element being responsive to said user control module for generating said patient information and for generating user display information for use by the user display system.

3. The apparatus of claim 1 further comprising:
said data acquisition module sends the plurality of the measurement data relating to a single disease to said data processing element.

4. The apparatus of claim 1 further comprising:
measuring modules, connected to said data acquisition module, comprising one or more of: one or more biomarker sensors, a pH sensor, a pO₂ sensor, a spectrometer, an optical sensor, and an electrical measurement system, a chemical measurement system, a physical measurement system, and an electrochemical measurement system.

5. A method for performing multiple diagnostics, the method comprising:
simultaneously, in terms of body functions, Measuring, using a data acquisition module, a plurality of parameters relating to diagnosis or treatment of a disease;
in response to said measured parameters, determining said diagnosis or treatment of said disease; and
effecting a treatment regimen based on said determined diagnosis or treatment.

6. The method of claim 5 further comprising:
selecting a plurality of the parameters to measure in response to a user input, and
displaying to said user, a diagnosis and a basis therefore.

7. The method of claim 5 further comprising:
in response to further measurements of said parameters, determining an effectiveness of said treatment, and modifying, as necessary, said treatment regimen; and
monitoring, on an on-going basis, the effectiveness of said treatment.

8. An apparatus for performing multiple diagnostics simultaneously, in terms of body function, comprising:
a data processing element;
a data acquisition module connected to the processing element;
a user control module connected to the processing element; and
said data acquisition module sending a plurality of measurement data simultaneously, in terms of body functions, to said data processing element, and in response thereto, the data processing element generates patient information.

9. The apparatus of claim 8 further comprising:
a treatment output module connected to the data processing element.

10. The method of claim 5 comprising:
in response to said further measurements of said parameters, modifying, as necessary, said treatment regimen.

11. An apparatus for simultaneously performing multiple diagnostics, comprising:
a diagnostic apparatus that performs multiple diagnostics on one or more diseases and that receives a plurality of inputs, wherein the diagnostic apparatus comprises:
one or more data acquisition modules that receive at least one of: biomarker data and data from one or more other measurements; and
one or more data processing modules connected to the one or more data acquisition modules that receive at least a portion of the data from the data acquisition module, wherein the data processing module responsively generates patient information relating to a patient based at least in part on the received portion of the data.

12. The apparatus of claim 11, wherein the data processing module transmits the patient information to one or more of: a treatment output module for effecting a treatment, an external database for storage, and an external display.

13. The apparatus of claim 11, further comprising a communications module that communicates with the external database for at least one of: receiving the patient information, storing the patient information, and managing the patient information.

14. The apparatus of claim 11, wherein the biomarker data is derived using spectroscopy.

15. The apparatus of claim 14, wherein the spectroscopy is selected from at least one of: fluorescent spectroscopy, mass spectroscopy, Raman spectroscopy, and Fourier transform infrared spectroscopy.

16. The apparatus of claim 11, wherein the data is measured in real time and simultaneously.

17. The apparatus of claim 11, wherein the one or more other measurements are received from at least one of: a pH sensor, a pO₂ sensor, an optical sensor, an electrical measurement system, a chemical measurement system, a physical measurement system, and an electrochemical measurement system.

18. The apparatus of claim 11, wherein a plurality of data acquisition modules receives the data from a plurality of inputs and transmits the portion of the received data to a plurality of data processing modules, and wherein each of the plurality of data processing modules transmits the portion of the data to a single processing module for subsequent processing.

19. The apparatus of claim 11, further comprising a wireless communications module that communicates with an external device.

20. The apparatus of claim 19, wherein the wireless communications module is configured to transmit the data and the generated patient information to the external device for displaying status of the patient.

21. The apparatus of claim 11, further comprising an external analysis device for diagnostic processing based on the generated patient information.

22. An apparatus for effecting treatments upon performing one or more diagnostics, comprising:
a treatment apparatus that effects a treatment regimen, wherein the treatment apparatus comprises:
a treatment output module connected to a data processing module that receives patient information, wherein the patient information is derived from at least one of: biomarker data and data from one or more other measurements and wherein the treatment output module generates treatment information based at least in part on the patient information; and
a feedback module connected to the data processing module that receives at least one of: updated biomarker data and updated data from the one or more other measurements, wherein the feedback module automatically controls at least one of drug delivery, electrical signal parameters, and treatment protocols in response to the updated data.

23. The apparatus of claim 22, wherein the biomarker data is derived from spectroscopy.

24. The apparatus of claim 23, wherein the spectroscopy is selected from at least one of: fluorescent spectroscopy, mass spectroscopy, Raman spectroscopy, and Fourier transform infrared spectroscopy.

25. The apparatus of claim 22, wherein the one or more other measurements are received from at least one of: a pH sensor, a pO₂ sensor, an optical sensor, an electrical measurement system, a chemical measurement system, a physical measurement system, and an electrochemical measurement system.

26. The apparatus of claim 22, further comprising a communications module that communicates with an external database for at least one of receiving the patient information, receiving the treatment protocols, receiving the treatment information, storing the patient information, storing the treatment information, managing patient information, managing the treatment information, and managing the treatment.

27. The apparatus of claim 26, wherein the communications are conducted using a secure protocol.

28. The apparatus of claim 26, wherein the external database provides at least a portion of the patient information and wherein the treatment output module uses the portion of the patient information for generating the treatment information.

29. The apparatus of claim 22, further comprising a wireless communications module that communicates with an external device.

30. The apparatus of claim 29, wherein the wireless communications module is configured to transmit at least one of: the data, the updated data, the patient information, and the generated treatment information for display.

31. The apparatus of claim 29, wherein the wireless communications module and the external device are configured to allow a user to manually control at least one of drug delivery, electrical signal parameters, and treatment protocols.
